(19) United States
(12) Patent Application Publication
(10) Pub. No.: US 2019/0339219 A1
(43) Pub. Date: Nov. 7, 2019

(54) ULTRASENSITIVE NITROGEN DIOXIDE GAS SENSOR BASED ON IRON NANOCUBES

(71) Applicant: OKINAWA INSTITUTE OF SCIENCE AND TECHNOLOGY SCHOOL CORPORATION, Kunigami-gun, Okinawa (JP)

(72) Inventors: Jerome Vernieres, Okinawa (JP); Stephan Steinhauer, Okinawa (JP); Mukhles Ibrahim Sowwan, Okinawa (JP)

(73) Assignee: OKINAWA INSTITUTE OF SCIENCE AND TECHNOLOGY SCHOOL CORPORATION, Kunigami-Gun, Okinawa (JP)

(21) Appl. No.: 16/312,579

(22) PCT Filed: Jun. 26, 2017
(86) PCT No.: PCT/JP2017/024566

§ 371 Date: Dec. 21, 2018

Related U.S. Application Data
(60) Provisional application No. 62/355,287, filed on Jun. 27, 2016.

(51) Int. Cl. G01N 27/12; G01N 33/00

(52) U.S. Cl. CPC G01N 27/127; B82Y 30/00; G01N 33/0037

(57) ABSTRACT

A gas sensor includes a substrate; a pair of electrodes facing each other on the substrate; and a plurality of metallic nanocubes each containing Fe, aggregated between the pair of electrodes and forming percolating paths between the pair of electrodes.

The present invention relates to gas sensors, and more particularly, to nitrogen dioxide gas sensors. This application claims the benefit of and hereby incorporates by reference U.S. Provisional Application No. 62/355,287, filed Jun. 27, 2016.

### TECHNICAL FIELD

### Background Art

The use of chemo-resistive gas sensors in exhaled breath analysis has recently attracted significant interest in biomedical applications. In particular, nitrogen oxides (NOx, mainly consisting of NO and NO₂) can be used as potential markers for early detection and diagnosis of diseases (NPL 1).

Breath analysis systems have been developed for asthma diagnosis, for instance, by applying highly sensitive NO₂ sensors in the ppb-level concentration range (NPL 2). For example, several metal oxides nanomaterials have been developed for NO₂ detection (NPLs 3-4), including Fe oxide nanoparticles (NPL 5).

### CITATION LIST

### Non Patent Literature

NPL 1: Ou, J., Z. et al., Physisorption-based charge transfer in two-dimensional SnS₂ for selective and reversible NO₂ gas sensing. ACS Nano. 9, 10313-10323 (2015).
NPL 2: Macagnano, A., Bearzotti, A., De Cesare, F. and Zampetti, E., Sensing asthma with portable devices equipped with ultrasensitive sensors based on electrospun nanomaterials. Electroanalysis 26, 1419-1429 (2014).
NPL 3: Zhang, D., Liu, Z., Li, C., Tang, T., Liu, X., Han, S., Lei, B. & Zhou, C., Detection of NO₂ down to ppb levels using individual and multiple In₂O₃ nanowire devices. Nano Lett. 4, 1919-1924 (2004).
NPL 4: Oh, E., Choi, H.-Y., Jung, S.-H., Cho, S., Kim, J. C., Lee, K.-H., Kang, S.-W., Kim, J., Yun, J.-Y. & Jeong, S.-H., High performance NO₂ gas sensor based on ZnO nanorod grown by ultrasonic irradiation. Sens. Actuators B 141, 239-243 (2009).
NPL 5: Navale, S. T., Bandgar, D. K., Nalage, S. R., Khuspe, G. D., Chougule, M. A., Kolekar, Y. D., Sen, S. & Patil, V. B., Synthesis of Fe₂O₃ nanoparticles for nitrogen dioxide gas sensing applications. Ceram. Int. 39, 6453-6460 (2013).
NPL 6: Steinhauer, S. et al., Single CuO nanowires decorated with size-selected Pd nanoparticles for CO sensing in humid atmosphere. Nanotechnology 26, 175502 (2015).
NPL 7: Grammatikopoulos, P., Steinhauer, S., Vernieres, J., Singh, V. and Sowwan, M., Nanoparticle design by gas-phase synthesis. Advances in Physics: X 1, 81-100 (2016).
NPL 8: Zhao, J. et al., Formation mechanism of Fe nanocubes by magnetron sputtering inert gas condensation. ACS Nano. 10, 4684-4694 (2016).
NPL 9: Benelmekki, M. et al., A facile single-step synthesis of ternary multicore magneto-plasmonic nanoparticles. Nanoscale 6, 3532-3535 (2014).

### SUMMARY OF INVENTION

### Technical Problem

However, for a successful gas sensor technology commercialization and integration with integrated-circuit manufacturing, the development of scalable nanomaterial fabrication methods that are compatible with industrial complementary metal-oxide silicon (CMOS) technology (NPL 6) (without the inherent products introduced by chemical synthesis from precursors and surfactants) is of crucial importance.

An object of the present invention is to provide a new and improved gas sensor so as to obviate one or more of the problems of the existing art.

### Solution to Problem

To achieve these and other advantages and in accordance with the purpose of the present invention, as embodied and broadly described, in one aspect, the present invention provides a gas sensor, comprising: a substrate; a pair of electrodes facing each other on the substrate; and a plurality of metallic nanocubes each containing Fe, aggregated between the pair of electrodes and forming percolating paths between the pair of electrodes.

In the gas sensor described above, the nanocubes may be made of Fe.

In the gas sensor described above, the nanocubes may be made of FeAu.

In the gas sensor described above, the pair of electrodes may be interdigitated electrodes.

In the gas sensor described above, at least some of the plurality of the nanocubes may have lateral widths of less than 50 nm.

In the gas sensor described above, at least some of the plurality of the nanocubes may have lateral widths of less than 15 nm.

In the gas sensor described above, at least some of the plurality of the nanocubes may have lateral widths of less than 10 nm.

In the gas sensor described above, the pair of electrodes may be made of Au.

### Advantageous Effects of Invention

According to one or more aspects of the present invention, it becomes possible to provide efficient, reliable, and accurate gas sensors.

Additional or separate features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

It is to be understood that both the foregoing general description and the following detailed description are exemplary and explanatory, and are intended to provide further explanation of the invention as claimed.

### BRIEF DESCRIPTION OF DRAWINGS

FIG. 1, shows a schematic representation of a high-vacuum magnetron sputtering inert-gas aggregation system used in making embodiments of the present invention.

In FIG. 2, (a) shows HAADF-STEM Z-contrast image of a typical Fe/Fe oxide core-shell nanocube and the corresponding EELS line-scan profile, the shell (outer part) and the core (center part). (b) shows a near-edge fine structure of the O—K edge (top graph) and Fe L_{2,3} edge (bottom graph). (c) shows an HRTEM image along [100] zone axis and corresponding FFT of the core and the (core+shell) shown in (d) and (e), respectively.

In FIG. 3, (a) shows a low-magnification TEM micrograph of FeAu nanocubes. The upper left inset is high-resolution TEM image of a representative single-crystalline nanocube. (b) shows EDX scan and corresponding EDX line scan profile (upper right inset) over one FeAu nanocube. (c) shows a comparative normalized magnetization at room temperature for the Fe and FeAu nanocubes in aqueous solution. The inset shows temperature dependence of the coercive field. (d) shows UV-vis absorbance spectra for Fe and FeAu nanocubes.

In FIG. 4, (a) shows a schematic representation of the magnetron sputtering source used for Fe NP synthesis to fabricate a Fe-based gas sensor device according to an embodiment of the present invention. (b) is a low-magnification transmission electron microscope (TEM) image showing the Fe nanocubes with their corresponding size distribution. (c) shows a scanning electron microscope (SEM) image of the electrode device (left image) covered with a percolating film of Fe nanocubes (right image). (d) shows the resistance change of the gas sensor during exposure to ppb-level NO₂ concentrations (operation temperature 200° C.).

FIG. 5 shows high-resolution scanning TEM image of Fe nanocube (a) before the experiment, (b) after in situ thermal oxidation (200° C., 1 h, 20 mbar O₂), and (c) after ex situ control experiment (200° C., 1 h, ambient air). (d) shows a low magnification TEM image of Fe nanocubes after in situ thermal oxidation and a specifically chosen area of low cubic purity (square).

### DESCRIPTION OF EMBODIMENTS

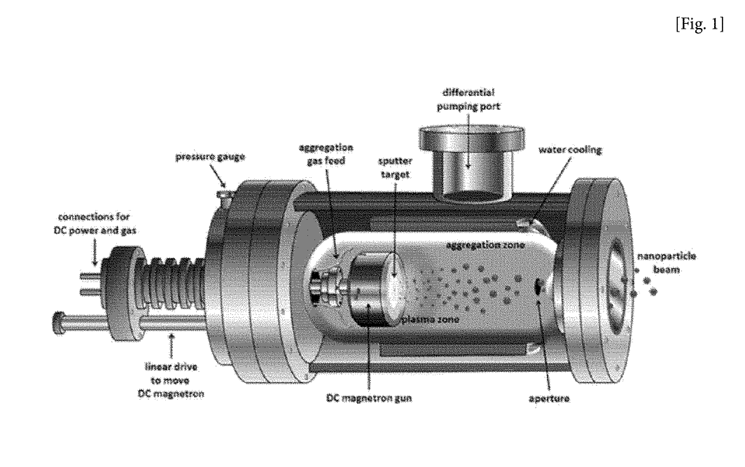

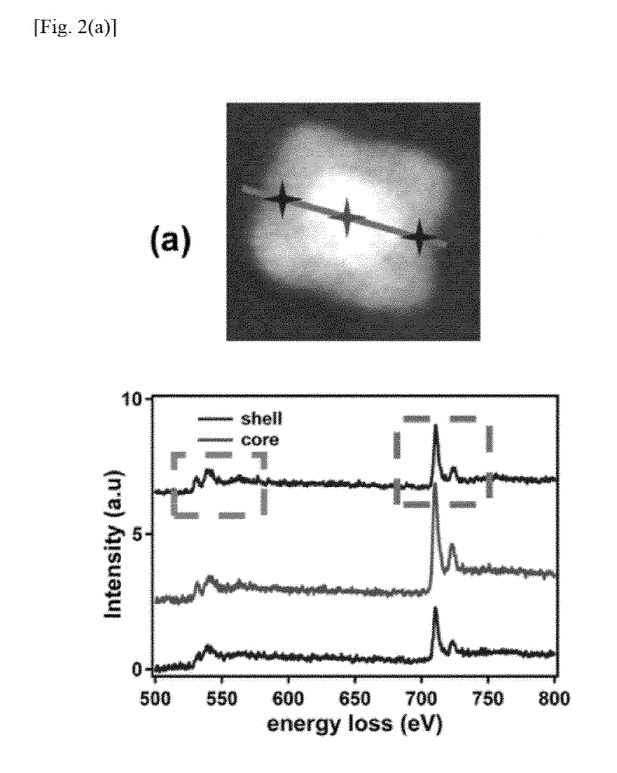

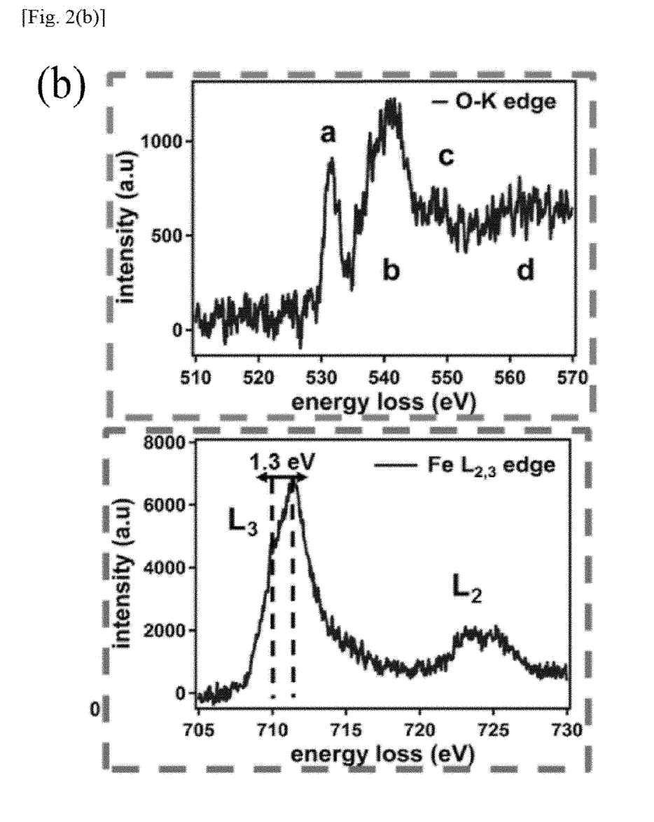

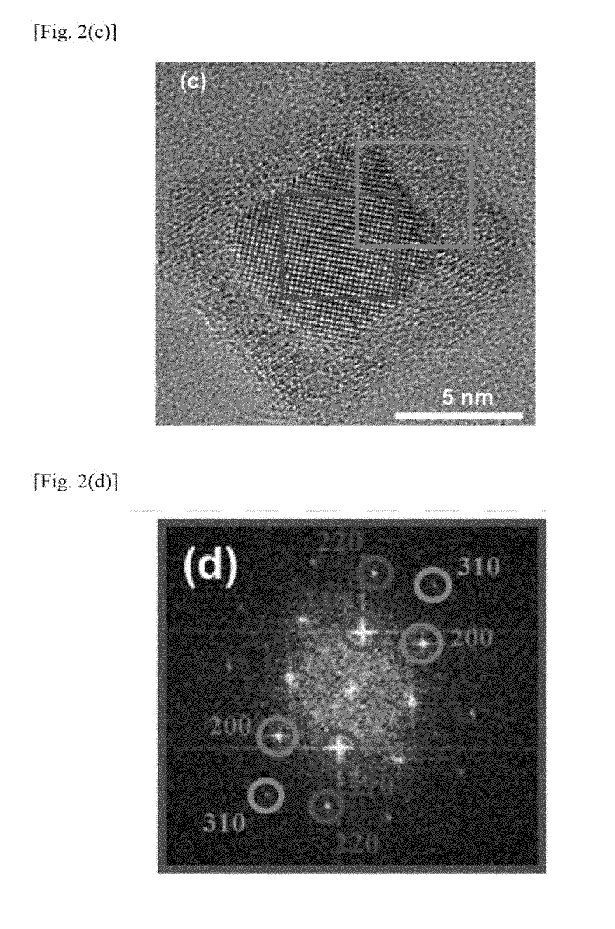

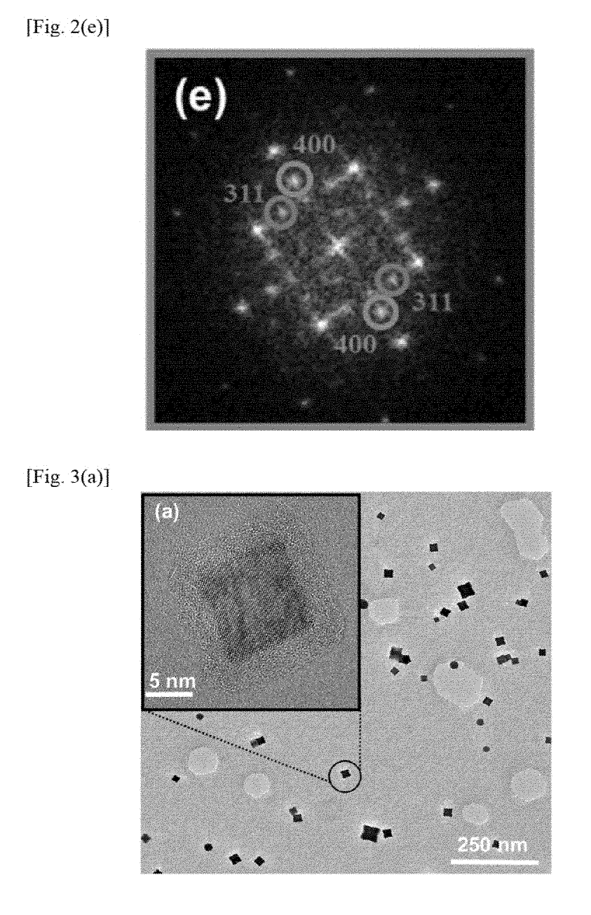

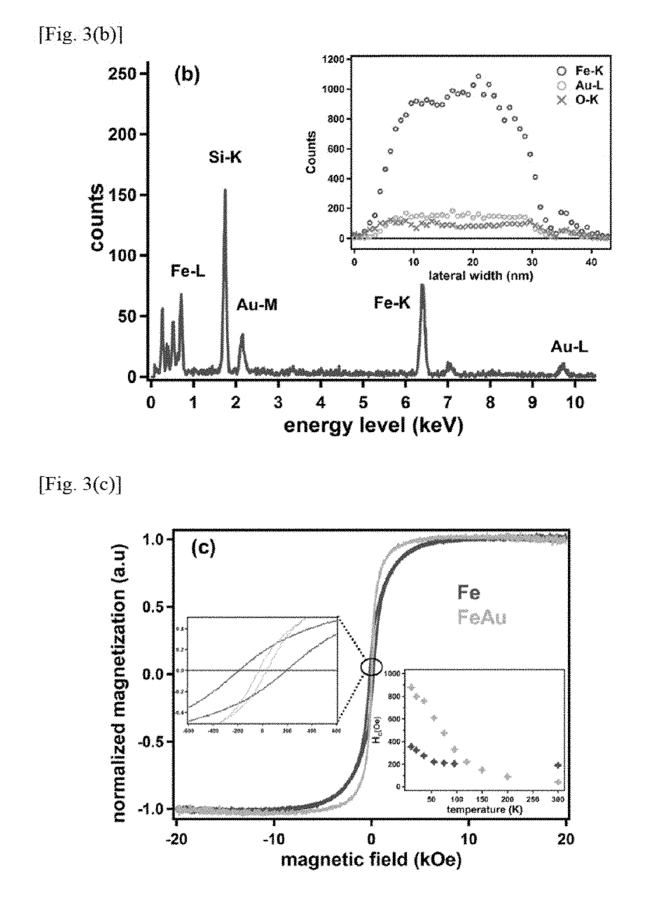

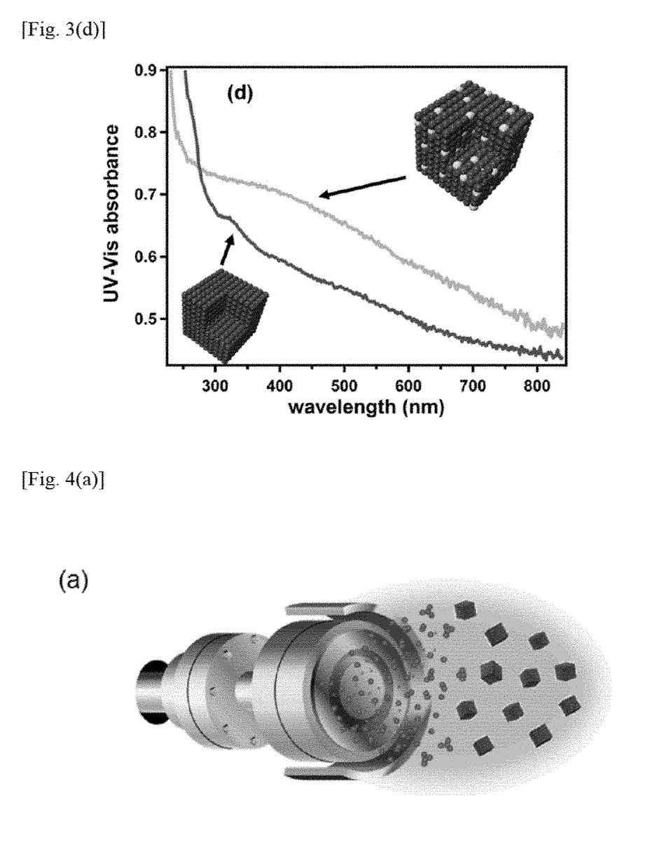

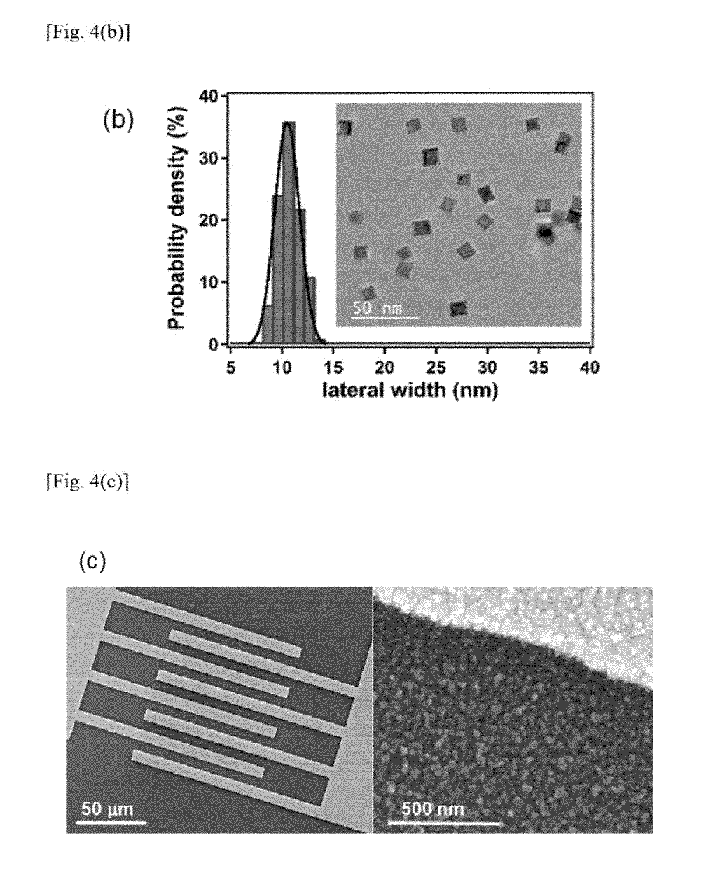

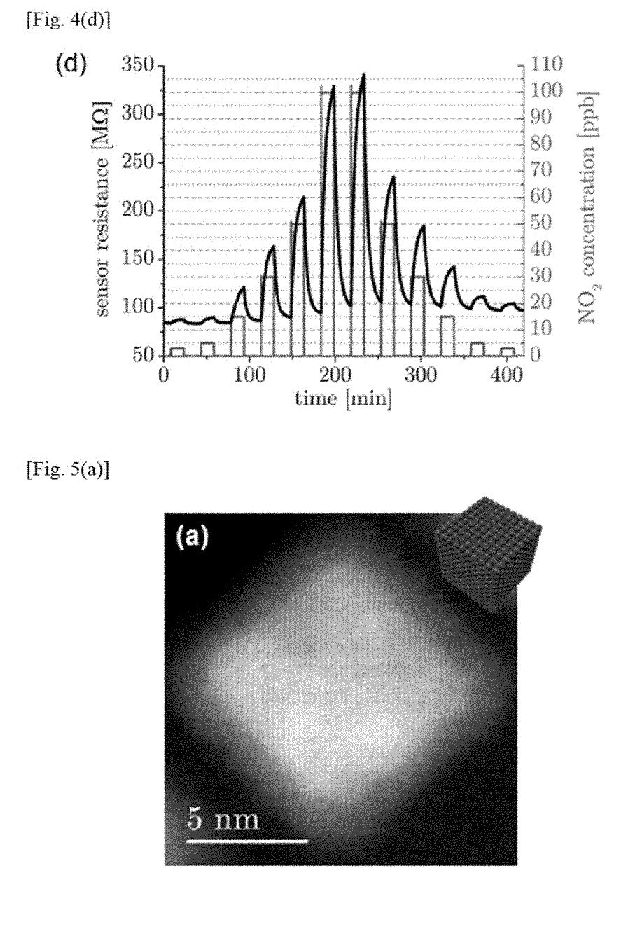

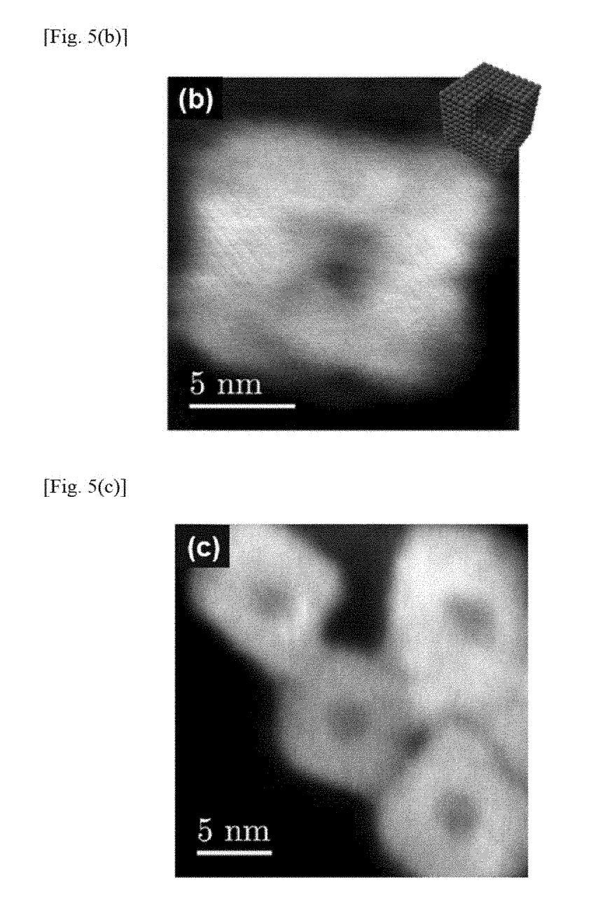

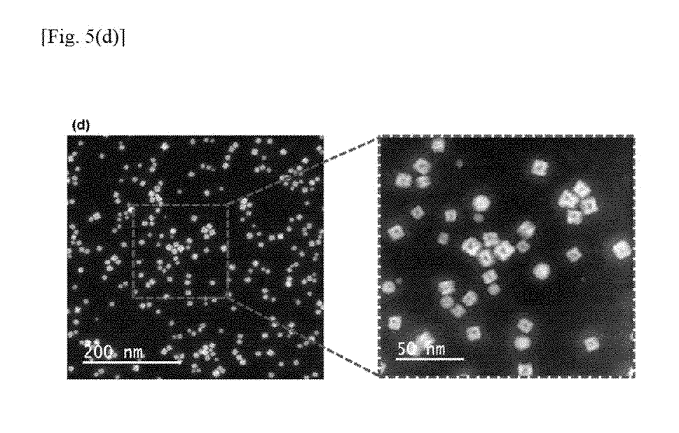

The present disclosure presents, in one aspect, an ultrasensitive (ppb level) NO₂ gas sensor based on a percolating film of Fe nanocubes. Fe nanocubes have been synthesized using a magnetron sputtering inert-gas condensation apparatus, as FIGS. 1 and 4(a) (NPLs 7-9). The method and experimental setup is described in NPLs 7-9. Prior to sputtering, the base pressures were kept below 10⁻⁶ mbar and 10⁻⁸ mbar for the aggregation chamber and the main chamber, respectively. For the experiments, an Argon (Ar) flow of 55 sccm was introduced in the aggregation chamber in order to maintain a pressure differential between the two chambers, which dictates the residence time and temperature balance in the aggregation zone, and therefore the crystallinity and the size of the NPs. Pure Fe nanoparticles were initially formed through a supersaturated vapor of metal atoms by DC sputtering of a high-purity Fe target (99.9%). The DC power was adjusted to 100 W, the aggregation length was set to 90 mm and the substrate was rotated during deposition at 2 rotation per minute (rpm) to improve the uniformity. Well-defined Fe nanoparticles with controlled size and shape (high-purity of cuboid morphology) were achieved (see exemplary sample in FIG. 4(b) with 10.5 nm mean diameter and 7% standard deviation) and deposited on interdigitated Au electrodes (8 μm gap distance; see FIG. 4(c)) realized by photolithographic lift-off techniques on Si substrates covered with 300 nm thermal SiO₂. The assembly of Fe nanocubes to percolating films is shown in the right image of FIG. 4(c). Gas sensing measurements were performed in a commercial probe station (Advanced Research Systems). Gas mixtures were supplied using gas feedthroughs connected to a gas delivery system by adjusting flow rates of synthetic air and diluted NO₂ (5 ppm in N₂) using mass flow controllers (Bronkhorst). The sensor was pre-treated at a sample stage setpoint temperature of 300° C. for three hours in dry synthetic air and subsequently stabilized at a sample stage setpoint temperature of 200° C. FIG. 4(d) shows resistance changes of the Fe nanocubes film in dry synthetic air during exposure to pulses of NO₂ (concentration range 3-100 ppb) at a constant voltage bias of 0.5 V. As can be seen, NO₂ could be clearly detected in the investigated concentration range. Thus, the presented Fe nanocubes can be potentially utilized in exhaled breath analysis systems for asthma diagnosis.

### <Thermal Environment Effect on Nanoparticle Morphology>

FIG. 1 shows a schematic representation of a high-vacuum magnetron sputtering inert-gas aggregation system used in making embodiments of the present invention. In a magnetron sputtering inert-gas aggregation system, the sputter target is placed on the magnetron gun, and when aggregation gas (usually Ar) is fed into the chamber, plasma is formed by ionization due to electrical discharge (FIG. 1). Subsequently, Ar⁺ ions bombard the target, sputtering atoms off its surface. Unlike conventional sputtering, these energetic atoms collide with room-temperature Ar atoms, cool down, and, upon collisions with each other, eventually form nanoclusters.

There is a direct correlation between the morphology of the resultant clusters and the thermal environment in which they were generated. NP temperature during growth is governed by the relative rates between collisions with Ar and sputtered atoms; any variation in these rates may result in clearly distinct NP structures.

Besides their effect on size, subtle differences in the thermal environment can also have a major effect on the shape of the NPs. The rate of atomic deposition on the growing nanoclusters in combination with their current temperature can determine the morphology; decisive differences in kinetic growth modes give rise specifically to cubic rather than near-spherical shapes. Due to the sensitive correlation between growth conditions and the resultant nanoparticles, in what follows special emphasis is given to determining the former as accurately as possible in order to predict and control the properties of the latter.

### <Transmission Electron Microscopy Characterization of Fe Nanocubes>

In FIG. 2, (a) shows HAADF-STEM Z-contrast image of a typical Fe/Fe oxide core-shell nanocube and the corresponding EELS line-scan profile, the shell (outer part) and the core (center part). (b) shows a near-edge fine structure of the O—K edge (top graph) and Fe L_{2,3} edge (bottom graph). (c) shows an HRTEM image along [100] zone axis and corresponding FFT of the core and the (core+shell) shown in (d) and (e), respectively. Shape, crystalline structure, and uniformity of the Fe nanocubes were characterized using (scanning) TEM, high-resolution TEM (HRTEM), and electron energy-loss spectroscopy (EELS). The low-magnification high-angle annular dark-field (HAADF) scanning TEM images show well-defined and uniform Fe nanocubes with a distinct core/shell structure typical for metallic NPs covered by an oxide (air exposure at room temperature). Moreover, two distinctive morphologies depending on NP size were observed. The EELS line-scan profile along a representative Fe core/shell nanocube (FIG. 2, (a)) points to the presence of a metallic Fe core (Fe L_{2,3} edge at 711.7 eV) and a homogeneously distributed Fe oxide shell (O—K edge at 531.7 eV). The near-edge fine structure was characterized using a monochromator (energy resolution around 0.2 eV), which is shown in FIG. 2, (b). The O—K edge reveals the four distinct features (a-d) characteristic for the Fe oxide phase. The intensity ratio of the prepeak (a) compared to the major contribution (b) suggests the presence of either Fe₃O₄ and/or γ-Fe₂O₃ instead of the FeO phase. In addition, the near-edge fine structure of the Fe L_{2,3} edge shows the characteristic L₃ and L₂ white lines of Fe. Interestingly, we observe a specific splitting of the L₃ (1.3 eV) and L₂ white lines. This splitting is often related to an octahedral coordination of the Fe(III) species and is usually attributed to the γ-Fe₂O₃ phase.

The crystalline structure of the obtained Fe nanocubes was characterized using HRTEM imaging (FIG. 2, (c)). The fast Fourier transform (FFT) analysis of the core in FIG. 2, (d) revealed the (110), (200), and (310) reflections characteristic of the [001] zone axis for a bcc structure (α-Fe phase). Concerning the Fe oxide shell, the FFT (FIG. 2. (e)) demonstrated that the oxide is composed of an inverse spinel structure, which can be either γ-Fe₂O₃, Fe₃O₄, or an intermediate phase. A gradual decrease of the calculated lattice parameter toward a value close to that of γ-Fe₂O₃ phase was observed compared with that of the large nanocubes, which confirms the EELS results shown above.

### <Synthesis of Magneto-Plasmonic Fe—Au Nanocubes>

In FIG. 3, (a) shows a low-magnification TEM micrograph of the FeAu nanocubes. The upper left inset is high-resolution TEM image of a representative single-crystalline nanocube. (b) shows EDX scan and corresponding EDX lines can profile (upper right inset) over one FeAu nanocube. (c) shows a comparative normalized magnetization at room temperature for the Fe and FeAu nanocubes in aqueous solution. The inset shows temperature dependence of the coercive field. (d) shows UV-vis absorbance spectra for Fe and FeAu nanocubes.

It should be stressed that, following our deposition approach, uniformity in shape, size, and crystallinity is not compromised by simultaneous co-sputtering of nonmagnetic dopants. Hence, one can also tailor the chemical composition of bimetallic nanocubes to engineer multifunctional nanomaterials, such as magneto-plasmonic nanoalloys for biosensing, magnetic-resonance imaging contrast agents, hyperthermia, etc. As an additional advantage, the nonequilibrium nature of the growth process can lead to metastable final products with desirable properties.

As an example, the Fe—Au system, which combines the physical and chemical properties of its two constituent elements, is a promising candidate for numerous applications. The limited miscibility of Fe and Au normally implies a tendency of Au segregation owing to its positive heat of mixing. As a result, the vast majority of studies on the system focus on bifunctional, segregated structures, such as Fe—Au core-shell, dumbbell-like Au—Fe₃O₄, or star-sphere Au—Fe nanoparticles that simultaneously maintain the high saturation magnetization of Fe and red-shift the absorption peak of Au to the near infrared. On the other hand, the nanoalloy configuration also displays promising magneto-optical properties for various applications, due to the high spin-orbit coupling characteristics of Au. However, only a limited number of studies on the synthesis of Fe—Au nanoalloys have been reported to date, mostly by chemical methods, without conclusive results regarding the homogeneity of the nanoparticles.

Here, using gas phase synthesis from a composite Fe target with inserted Au pellets, the present inventors fabricated well-defined FeAu nanocubes (see FIG. 3, (a)) with single crystalline cores, as shown in FIG. 3, (b). FFT analysis indicates a single-phase bcc structure (α-Fe) with an expansion of the lattice parameter of about 3%-4%, which can be attributed to a purely substitutional solid solution with Au concentration of about 10%-15%, as confirmed by an energy dispersive X-ray spectroscopy (EDS) analysis of multiple nanocubes. Moreover, using EDS combined with EDS linescan profiling indicates the presence of both elements homogeneously distributed in the core, as shown in FIG. 3, (c). The FeAu nanocubes were dispersed in ultrapure water using a harvesting procedure (see details in the experimental section) based on a biocompatible polymer coating, polyvinylpyrrolidone (PVP). Their normalized magnetization in aqueous solution as a function of the applied magnetic field, M(H), is shown in FIG. 3, (d). A typical ferromagnetic behavior at room temperature is observed for the Fe and FeAu nanocubes with a coercive field (Hec) of 2000 and 400 e, respectively (left inset FIG. 3, (d)). The decrease of Hc in the FeAu sample can be attributed to weak dipolar interaction due to a lower particle density in the aqueous solution. In contrast, at low temperature, an inverse tendency is observed with an increase of the remanence accompanied by a drop of the coercively in the Fe sample (right inset FIG. 3, (d)), which confirmed the higher particle density on this sample. The optical properties of the Fe-based nanocubes were determined using UV-vis absorption spectroscopy (FIG. 3, (d)). FeAu nanocubes reveal a broadband absorption centered at about 450 nm compared to the Fe nanocubes, which show absorption at about 320 nm. The broadband absorption and blue shift (compared to the usual Au plasmon peak) obtained in the FeAu sample can be attributed to the good dispersion and homogeneity of the nanocubes in water solution, whereas the rather weak absorption band is expected due to the relatively low concentration of Au (compared to previous studies using Au-rich samples).

The present inventors' goal in growing homogeneous solid solution FeAu nanocubes was twofold: first, we explored the possibility for adding extra functionalities to our Fe nanocubes by doping with other metals. Also, the potential of our fabrication method for overcoming thermodynamic limitations was demonstrated in both physical and chemical ordering. Naturally, once a metastable configuration with an optimized composition is obtained, it can be reverted to an energetically favorable one by thermally assisted segregation processes, thus paving the way for future studies on tailored magneto-plasmonic nanostructures.

### <Chemoresistive Gas Sensing Application>

As described above, as an embodiment of the present invention, by employing our efficient synthesis of homogeneous Fe NPs, Fe nanocubes were assembled into percolating films on a device having interdigitated electrodes (see schematic illustration in FIG. 4, (c)) and their application for chemoresistive gas sensors was evaluated. In FIG. 4, (a) shows a schematic representation of the magnetron sputtering source used for Fe NP synthesis to fabricate a Fe-based gas sensor device according to the embodiment of the present invention. (b) is a low-magnification transmission electron microscope (TEM) image showing the Fe nanocubes with their corresponding size distribution. (c) shows a scanning electron microscope (SEM) image of the electrode device (left image) covered with a percolating film of Fe nanocubes (right image). (d) shows the resistance change of the gas sensor during exposure to ppb-level NO₂ concentrations (operation temperature 200° C.).

As explained below, ultrasensitive (ppb level) NO₂ gas sensors were achieved based on a percolating film of Fe nanocubes. Fe nanocubes have been synthesized using a magnetron sputtering inert-gas condensation method described above, as schematically illustrated in FIG. 4, (a). More details of the method and experimental setup is described in NPLs 7-9. Prior to sputtering, the base pressures were kept below 10⁻⁶ mbar and 10⁻⁸ mbar for the aggregation chamber and the main chamber, respectively. For the manufacture, an Argon (Ar) flow of 55 sccm was introduced in the aggregation chamber in order to maintain a pressure differential between the two chambers, which dictates the residence time and temperature balance in the aggregation zone, and therefore the crystallinity and the size of the NPs. Pure Fe nanoparticles were initially formed through a supersaturated vapor of metal atoms by DC sputtering of a high-purity Fe target (99.9%). The DC power was adjusted to 100 W, the aggregation length was set to 90 mm and the substrate was rotated during deposition at 2 rotation per minute (rpm) to improve the uniformity. Well-defined Fe nanoparticles with controlled size and shape (high-purity of cuboid morphology) were achieved (see exemplary sample in FIG. 4, (b)) with 10.5 nm mean diameter and 7% standard deviation) and deposited on interdigitated Au electrodes (8 μm gap distance; see FIG. 4, (c)) that were formed by photolithographic lift-off techniques on Si substrates covered with 300 nm thermal SiO₂. The assembly of Fe nanocubes to percolating films is shown in the right image of FIG. 4, (c). Gas sensing measurements were performed in a commercial probe station (Advanced Research Systems). Gas mixtures were supplied using gas feedthroughs connected to a gas delivery system by adjusting flow rates of synthetic air and diluted NO₂ (5 ppm in N₂) using mass flow controllers (Bronkhorst). The sensor was pre-treated at a sample stage setpoint temperature of 300° C. for three hours in dry synthetic air and subsequently stabilized at a sample stage setpoint temperature of 200° C. FIG. 4, (d) shows resistance changes of the Fe nanocubes film in dry synthetic air during exposure to pulses of NO₂ (concentration range 3-100 ppb) at a constant voltage bias of 0.5 V. As can be seen, NO₂ could be clearly detected in the investigated concentration range. Thus, the presented Fe nanocubes can be utilized in exhaled breath analysis systems for asthma diagnosis.

The conduction model of film-based devices and thus their sensor performance is strongly dependent on layer geometry and grain morphology. Traditionally, studies on gas sensitive materials are restricted to structural characterization of the employed nanostructures before sensor operation. However, this neglects the fact that elevated temperatures and oxidizing/reducing gas atmospheres can have a significant impact on the nanoscale morphology of the sensor device. In order to understand the gas sensing functionality of the presented Fe nanocubes, the present inventors utilized in situ experiments in an environmental TEM as a novel approach for assessing structural changes of gas-sensitive nanomaterials induced by elevated temperatures and oxidizing gas atmosphere. A high-resolution scanning TEM image of an Fe nanocube after ambient air exposure is presented in FIG. 5, (a), showing Fe—Fe oxide core-shell morphology, as described above. In situ thermal oxidation was performed (200° C., 1 h, 20 mbar O₂) resulting in distinct morphological changes of Fe nanocubes: formation of voids was observed in the NP center, as shown in FIG. 5, (b). This phenomenon is attributed to the Kirkendall effect-a difference in solid-state diffusion rates in alloying or oxidation reactions. The outward diffusion of metallic Fe is expected to be faster than the inward oxygen diffusion, resulting in consumption of the Fe core and void formation. Our environmental TEM results demonstrate that Fe NPs retain close-to-cubic shapes after complete thermal oxidation, which differs from literature results on Fe nanocubes that adopt approximately spherical shapes after long-term storage at room temperature.

In an ex situ control experiment shown in FIG. 5, (c), equivalent morphological changes of Fe nanocubes were observed, validating the environmental TEM results and suggesting that the gas sensor resistance is determined by the percolation of fully oxidized, hollow Fe oxide nanocubes. We attribute the excellent sensing performance to the morphology of individual NPs: the space charge region due to chemisorbed gas species is expected to extend to the entire Fe oxide shell, as the Debye length of undoped metal oxide semiconductors is typically in the range of a few nm. Thus, the formation of hollow nanostructures ensures optimized NP morphologies for gas sensing, resulting in electrical conductivity being highly sensitive to minute NO₂ concentrations. It is noteworthy that void formation via the Kirkendall effect is mostly suppressed in spherical NPs under these specific conditions, as shown in FIG. 5, (d), once more emphasizing the importance of using anisotropic NP shapes such as nanocubes. As magnetron sputtering inert-gas condensation has been successfully employed for the synthesis of diverse nanoparticle structures, it can be applied for a broad range of materials, aiming at morphological control of gas sensing activity.

In summary, the present disclosure provides a novel miniaturized chemo-resistive nitrogen dioxide (NO₂) gas sensor suitable for biomedical applications such as asthma detection. One of the novelties of this invention lies in engineering highly faceted Fe nanocubes and the integration of these nanocubes in the form of high surface area porous thin film between metal electrodes using a gas-phase CMOS (complementary metal-oxide silicon) compatible method. This low cost thin film allows detection of very low concentrations (ppb level) of NO₂ gas. In particular, multifunctional Fe-based nanocubes were synthesized by a simple and versatile gas-phase method. The excellent sensing properties due to specific NP morphologies combined with the inherent advantages of NP gas phase synthesis make this approach a highly promising candidate for large-scale production of miniaturized, high-performance gas sensor devices integrated with standard microelectronic components. Furthermore, we tune the magneto-plasmonic properties by introducing dopant materials in hybrid FeAu nanocubes, which opens new prospects for biomedical applications as well as for future studies on chemoresistive sensors with improved selectivity.

### <Additional Details of Experiments/Fabrications>

Synthesis of Fe NPs: Fe NPs were prepared by a commercial inert-gas condensation magnetron sputtering source. The aggregation chamber was water-cooled and the base pressure was kept below 10⁻⁶ mbar prior to sputtering. In all fabrications, an argon (Ar) flow of 55 sccm was set to maintain a similar differential pressure, which dictates the residence time and temperature balance in the aggregation zone, and therefore the crystallinity and the size of the nanoparticles. Pure Fe NPs were initially formed through a supersaturated vapor of metal atoms by DC sputtering of a high-purity Fe target (99.9%) under Ar atmosphere. The aggregation length was set to 90 mm and the substrate was rotated during deposition at two rotations per minute (rpm) to improve the uniformity.

Synthesis of FeAu NPs: FeAu NPs were obtained using a modified Fe target with two Au pellets inserted at positions within the expected racetrack. The NPs were deposited on TEM grids and on a PVP film to allow for their transfer in aqueous solution. For the PVP film, a glass slide substrate (76 mm×26 mm) was thoroughly cleaned in dry methanol for 10 min under ultrasonication, then dried under N₂ gas. 10 mg of PVP (Sigma-Aldrich, St. Louis, USA) were dissolved in 250 μL of methanol solution and gently dispensed onto the clean glass substrate. A thin PVP film was formed by a spin-coater (MS-A-150, MIKASA, Japan) operated at 3000 rpm for 30 s. NPs were exfoliated by immersing the NPs/PVP/glass samples in methanol and sonicating for 15 min, followed by a separation step to remove the excessive PVP polymer using a centrifuge at 100 000 rpm for 60 min. After washing the precipitated NPs with methanol, the NPs were re-dispersed in ultrapure water from a Milli-Q system (Nihon Millipore K.K., Tokyo, Japan) using 0.1 μm filters.

Materials Characterization: The Fe NPs were deposited on Si substrate (5 mm×5 mm) and Si₃N₄ amorphous TEM grids (8 mm film, 60 mm×60 mm Apert. on 5 mm×5 mm windows) for characterization after exposure to air. Nanoparticle dispersions on Si substrates and on gas sensing devices were analyzed using an FEI Quanta FEG 250 scanning electron microscope. HRTEM images were acquired using an FEI Titan 80-300 kV environmental TEM equipped with a Cs-image corrector and operated at 300 and 80 kV. Particle size distributions of Fe nanocubes were determined by measuring the lateral dimensions of more than 1000 nanoparticles for each sample using low magnification TEM images. EELS was performed to study the native oxide formed on individual Fe nanocubes in scanning transmission electron microscopy (STEM) mode at 80 kV (energy resolution of 0.2 eV estimated using the full-width at half maximum of the zero-loss peak and a collection semi-angle around 13 mrad). The energy loss spectra of the O—K edge and Fe L2,3-edge were acquired simultaneously in dual EELS mode.

In Situ Measurements: Environmental TEM studies were performed using a commercial TEM heating holder based on heating chips with closed loop temperature control (Protochips Inc.). Fe NPs were imaged on a carbon support in STEM mode using an HAADF detector. In situ thermal oxidation was performed with a heater setpoint temperature of 200° C. for 1 h at a pressure of 20 mbar O₂. Ex situ control experiments were performed by heating Fe NPs on Si₃N₄ TEM grids to 200° C. for 1 h in ambient air.

It will be apparent to those skilled in the art that various modification and variations can be made in the present invention without departing from the spirit or scope of the invention. Thus, it is intended that the present invention cover modifications and variations that come within the scope of the appended claims and their equivalents. In particular, it is explicitly contemplated that any part or whole of any two or more of the embodiments and their modifications described above can be combined and regarded within the scope of the present invention.

1. A gas sensor, comprising:
a substrate;
a pair of electrodes facing each other on the substrate; and
a plurality of metallic nanocubes each containing Fe, aggregated between the pair of electrodes and forming percolating paths between the pair of electrodes.

2. The gas sensor according to claim 1, wherein the nanocubes are made of Fe.

3. The gas sensor according to claim 1, wherein the nanocubes are made of FeAu.

4. The gas sensor according to claim 1, wherein the pair of electrodes are interdigitated electrodes.

5. The gas sensor according to claim 4, wherein nanocubes are made of Fe.

6. The gas sensor according to claim 4, wherein nanocubes are made of FeAu.

7. The gas sensor according to claim 1, wherein at least some of the plurality of the nanocubes have lateral widths of less than 50 nm.

8. The gas sensor according to claim 1, wherein at least some of the plurality of the nanocubes have lateral widths of less than 15 nm.

9. The gas sensor according to claim 1, wherein at least some of the plurality of the nanocubes have lateral widths of less than 10 nm.

10. The gas sensor according to claim 1, wherein the pair of electrodes is made of Au.
